(19) United States
(12) Patent Application Publication
(10) Pub. No.: US 2023/0263944 A1
(43) Pub. Date: Aug. 24, 2023

(54) DUAL FUNCTION SURFACE FOR CELL CAPTURE AND SPREADING

(71) Applicants: THE ROYAL INSTITUTION FOR THE ADVANCEMENT OF LEARNING/MCGILL UNIVERSITY, Montréal (CA); UNIVERSITÉ LAVAL, Québec (CA)

(72) Inventors: Corinne A. HOESLI, Montréal (CA); Omar BASHTH, Montréal (CA); Mohamed Aly ELKHODIRY ABOELEELA MOHAME, Westmount (CA); Gaétan LAROCHE, Québec (CA); Manève BOULANGER, Montréal (CA)

(21) Appl. No.: 18/005,204

(22) PCT Filed: Jul. 14, 2021
(86) PCT No.: PCT/CA2021/050968

§ 371 Date: Jan. 12, 2023

Related U.S. Application Data
(60) Provisional application No. 63/051,608, filed on Jul. 14, 2020.

(51) Int. Cl. A61L 27/54; C07K 16/28; C12N 11/06

(52) U.S. Cl. CPC A61L 27/54; C07K 16/2896; C12N 11/06; A61L 2300/25; A61L 2300/256

(57) ABSTRACT

There is provided a surface functionalized with cross linking groups adapted to receive antibodies and/or fragments thereof. The surface has an antibody binding biomolecule having a linker region which is covalently crosslinked to functional groups and an antibody binding region. The surface also has a cell interacting biomolecule having a linker region which is covalently crosslinked to functional groups of the surface and a cell interacting region that imparts functional attributes including cell adhesion, spreading, proliferation, differentiation and/or a functional response. The two biomolecules are present in independently controlled concentrations and have similar small molecular weights.

### CROSS-REFERENCE TO RELATED APPLICATION

The present application is claiming priority from U.S. Provisional Application No. 63/051,608 filed Jul. 14, 2020, the content of which is hereby incorporated by reference in its entirety.

### TECHNICAL FIELD

This disclosure generally relates to the field of functionalized surfaces for cell capture and expansion.

### BACKGROUND OF THE ART

Medical implants surfaces and ex vivo tissue culture surfaces often lack necessary biological molecules needed to maximize cell numbers and specificity on the surfaces. To improve their performance, the surfaces have been modified by grafting different biomolecules such as extracellular matrix (ECM) proteins, antibodies, growth factors, and peptides. Surfaces with immobilized antibodies, for example, have shown immense potential in selecting cell types and capturing them on the surface based on their surface antigens which have since then been utilized for vascular stents and cell purification systems. Surfaces modified with ECM-derived peptides were shown to promote cell adhesion, proliferation, and differentiation making it ideal for tissue engineering scaffolds and cell expansion platforms. However, each of these types of molecules lack the ability to carry certain integral functions needed to successfully recruit selected cell subtypes and promote their subsequent spreading, proliferation and/or functionality.

Therefore, improvements are desired in the functionalization of surfaces to ameliorate cell capture, survival and function.

### SUMMARY

In a first aspect, there is provided a surface functionalized with cross linking groups, the surface adapted to receive antibodies and/or fragments thereof that bind a specific subtype of cells capturing the cells onto the functionalized surface and/or that bind cellular products, the surface comprising: an antibody binding biomolecule comprising a linker region which is covalently crosslinked to functional groups on the surface and an antibody binding region that binds to any non-variable region of the antibodies or the fragments thereof and controls the orientation of the antibodies and/or the fragments thereof such that the variable regions are away from the surface, and the antibody binding biomolecule having a molecular weight of less than 10,000 g/mol; and a cell interacting biomolecule comprising a linker region which is covalently crosslinked to functional groups of the surface and a cell interacting region, the cell interacting biomolecule has a three dimensional structure such that the cell interacting region is oriented away from the surface when the linker region has crosslinked with the surface, and the cell interacting biomolecule has a molecular weight of less than 10,000 g/mol; wherein a concentration of the antibody binding biomolecule and a concentration of the cell interacting biomolecule on the surface are each independently controlled within a predetermined range that allows both the antibodies and/or the fragments thereof and the cell binding biomolecule to be functional.

In one embodiment, the molecular weight of the antibody binding biomolecule is between about 500 g/mol to about 2000 g/mol.

In one embodiment, the molecular weight of the cell interacting biomolecule is between about 500 g/mol to about 2000 g/mol.

In one embodiment, the antibody binding biomolecule further comprises one or more spacing regions comprising polyethylene glycol (PEG) and/or glycine.

In one embodiment, the cell interacting biomolecule further comprises one or more spacing regions comprising polyethylene glycol (PEG) and/or glycine.

In one embodiment, the cell interacting region of the cell interacting biomolecule comprises a peptide derived from extracellular matrix proteins.

In one embodiment, the antibody binding region of the antibody binding biomolecule comprises a RRGW peptide.

In one embodiment, the linker region of the antibody binding biomolecule comprises sulfosuccinimidyl 4-(n-maleimidophenyl)butyrate (Sulfo-SMPB).

In one embodiment, the linker region of the cell interacting biomolecule comprises Sulfo-SMPB.

In one embodiment, the cell interacting region of the cell interacting biomolecule comprises a RGD peptide.

In one embodiment, the surface is one of a flat surface, an interior cylindrical surface, an helix/screw-shaped material or an exterior surface of a microbead.

In one embodiment, the surface is a slide or multiwell plate.

In another embodiment, the surface is of polystyrene or of cobalt-chrome (CoCr).

In one embodiment, a ratio of the molecular weight of the antibody binding biomolecule to the molecular weight of the cell interacting biomolecule is between about 1:10 to about 10:1.

In one embodiment, the cells are cancer cells, primary cells or in vivo cells.

In one embodiment, the cells are endothelial progenitor cells such as endothelial colony-forming cells.

In a second aspect, there is provided a vascular stent device comprising the surface defined in the first aspect.

In a third aspect, there is provided a microcarrier comprising the surface defined in the first aspect.

In a fourth aspect, there is provided a method of producing a surface having a dual function of capturing cells and/or cell products and promoting proliferation, adhesion, spreading, differentiation and/or function of the cells, comprising: providing an activated surface having a crosslinking chemical group bound to one or more linker molecules; crosslinking an antibody binding biomolecule to the one or more linking arms at the linker region of the antibody binding biomolecule by adding a predetermined concentration of the antibody binding biomolecule onto the surface, the antibody binding biomolecule having an antibody binding region that binds to the Fc region of antibodies and/or the fragments thereof and controls the orientation of the antibodies and/or the fragments thereof such that the variable regions are away from the surface, and the antibody binding biomolecule having a molecular weight of less than 10,000 g/mol, and crosslinking a cell interacting biomolecule to the one or more linkers at the linker region of the cell interacting biomolecule by adding a predetermined concentration of the cell interacting biomolecule onto the surface, the cell interacting biomolecule having a cell interacting region that interacts with cellular receptors of the cells to promote the adhesion, spreading, proliferation, activation and/or function of the cells on the surface, a three dimensional structure such that the cell interacting region is oriented away from the surface when the linker region has crosslinked with the surface, and a molecular weight of less than 10,000 g/mol, thereby obtaining a conjugated surface; and incubating the conjugated surface with the antibodies and/or the fragments thereof and allowing the antibodies and/or the fragments thereof to bind the antibody binding biomolecule.

In one embodiment, prior to step a) an optimization step is performed to determine the predetermine concentration of the antibody binding biomolecule and the predetermined concentration of the cell interacting biomolecule such that the surface has an optimized dual function of both capturing the cells and promoting the proliferation, adhesion, and/or spreading of the cells.

In one embodiment, a first washing step is performed between step b) and step
c) and a second washing step after step c).

In one embodiment, prior to step a), activating an inert surface to obtain the activated surface.

### DESCRIPTION OF THE DRAWINGS

FIG. 1 is an enlarged schematic view of a surface functionalized with an antibody binding biomolecule and a cell interacting biomolecule.

FIG. 2 is a schematic view illustrating the state of the surface at various steps during the process for producing the surface.

FIG. 3 is a schematic view of a surface and its interaction with a cell from binding to spreading.

FIG. 4 shows surface functionalization and durability of RGD-TAMRA (CGK(PEG3-TAMRA)GGRGDS-NH2) surface modification via adsorption vs covalent conjugation, wherein it is depicted in (A) the structure of RGD-TAMRA-modified polystyrene surfaces using sulfo-SMPB as a bilinker for covalent conjugation; in (B) the fluorescence imaging of spots of RGD-TAMRA coatings that were prepared via either covalent conjugation (with sulfo-SMPB) or adsorption (without sulfo-SMPB) and imaged immediately after modification or 28 days later; and in (C) the quantification of the average fluorescence intensity of RGD-TAMRA spots after up to 28 days. *P<0.05; **P<0.01 with N=3.

FIG. 5 shows an immobilization of mouse anti-human CD105 on conjugated or adsorbed RRGW(PEG3)C spots on polystyrene substrates, and detected by AF488-F(ab′)2-goat anti-mouse; wherein in (A) the chemical structure of the covalent conjugation of RRGW peptide onto activated polystyrene surfaces with sulfo-SMPB is seen; in (B) the schematic representation of experimental design to immobilize and detect the grafted antibodies on the surface is illustrated; in (C) the fluorescence imaging of immobilized antibodies on either adsorbed (without sulfo-SMPB) or conjugated (with sulfo-SMPB) spots of RRGW (300 μM during the conjugation step) is shown; and in (D) the average of mean intensities of the detected antibodies on adsorbed or conjugated RRGW spots is illustrated, wherein the concentrations represent the amount of RRGW added during the conjugation or adsorption step, where 0 μM as a control represents the background regions without RRGW. Samples were compared to the control with **p<0.01, *p<0.05 and NS (Not Significant)>0.05, N=3 experimental replicates (6 spots analyzed per experimental replicate).

FIG. 6 shows the detection of TAMRA fluorophores on peptide-modified polystyrene surfaces using a novel direct ELISA assay, wherein in (A) a comparison between conjugated or adsorbed RGD-TAMRA, conjugated RGD (no TAMRA)— all peptides at a concentration of 20 μM in solution—with 1% BSA solution in phosphate-buffered saline (PBS) containing 0.05% Tween®-20 as a background control is seen; and in (B) a saturation curve of conjugated RGD-TAMRA on polystyrene substrate. **p<0.01 with N=3 experimental replicates is shown.

FIG. 7 shows a stability study of immobilized mouse anti-human CD105 on conjugated or adsorbed spots of RRGW applied to polystyrene surfaces incubated for 1 h statically or under 1.5 dyn/cm² shear stress flow, wherein antibodies were then detected by AF488-F(ab′)2-goat anti-mouse; and in (A) fluorescence imaging of antibodies on either conjugated RRGW incubated in PBS, 10% or 70% FBS in endothelial growth media −2 (EGM-2) or adsorbed RRGW incubated in PBS is seen; and in (B) an average of mean intensities detected and analyzed for only spot regions with background (no RRGVV) is considered as a control. *p<0.05, **p<0.01 and NS (Not Significant)>0.05; N=3 experimental replicates (6 spots analyzed per experimental replicate).

FIG. 8 is a schematic representation of the circumscribed central composite design (CCC) showing in (A) the normalized values of each point in the model, and in (B) the concentrations in solutions (in μM) of RGD-TAMRA and RRGW in X-axis and Y-axis, respectively.

FIG. 9 illustrates a central composite design of bifunctional polystyrene surface modification with antibodies and RGD-TAMRA, wherein in (A) a representative fluorescence imaging of several ratios combining anti-CD105 antibodies immobilized on conjugated RRGW, and RGD-TAMRA peptides is shown; and in (B) RGD-TAMRA and antibody surface responses regarding the different concentrations of both molecules, N=3 experimental replicates are illustrated.

FIG. 10 shows ECFC behavior after seeding on different surfaces under static conditions, where cell capture is not required for cell tethering to surfaces. As shown, RGD peptides increase cell spreading irrespective of antibody presence. Cells were stained for F-actin (red) and DAPI (blue). Images were used to quantify cell number and surface area per cell. Unmodified=aminated polystyrene surfaces without further modifications. **p<0.01, *p<0.05 with N=3 experimental replicas.

FIG. 11 shows an analysis of ECFC surface adhesion under dynamic flow conditions in a perfusion loop on modified polystyrene surfaces, where cell capture is required for cell tethering to surfaces. As shown, antibodies increase the number of captured cells irrespective of RGD peptide presence. The combined effects of antibodies on cell capture and RGD peptides on cell spreading result in significantly higher surface coverage on bi-functional surfaces compared to surfaces with antibodies alone or peptides alone. Cells were circulated over the surface for 2 and half hours at 1 dyn/cm² and cell number and spreading were analyzed using immunocytochemistry. **p<0.01, *p<0.05 with N=4 experimental replicas.

FIG. 12 shows surface-induced ECFC proliferation on bifunctional polystyrene surfaces. As shown, the proliferative effect of RGD on the cells is maintained even when antibodies are also present on the surface. (A) Representative images of proliferative ECFCs after 24 hours of seeding on the different surface conditions visualized by an EdU stain (green) and a blue nuclear stain. (B) Quantification of the percentage of Edu+ cells on the four different surfaces. *P<0.05 with N=3 experimental replicas from different ECFC donors.

FIG. 13 shows selectivity of ECFC capture on bifunctional polystyrene surfaces in the presence of PBMCs under dynamic flow. As shown, the capacity to capture cells by antibodies is maintained even when RGD peptides are also present on the surface. (A) Representative fluorescence images from the four different conditions immediately after a 1:1 mixture of PBMCs (pre-stained with Blue) and ECFCs (pre-stained with green) were circulated over the surfaces for 1 h in a microfluidic flow model at 1 dyn/cm². Flow direction was from right to left. B) Quantification of the amount of captured ECFCs as a percentage of all cells found on each condition. *P<0.05 with N=3 experimental replicas from different ECFC donors.

FIG. 14 shows microscopy images of 6 polystyrene well plates with HUVEC cells subject to different conditions after 3 h of shaker incubation. In this figure, antibodies were immobilized using cysteine-Protein G instead of using Fc-interacting peptides as described herein. The surface conditions are unmodified initial substrate (aminated polystyrene plate), RGD-TAMRA only, High anti-CD144 only (concentration of 5.5×10⁻⁶ M antibody in solution), collagen (adsorbed), RGD-TAMRA+high anti-CD144, and RGD-TAMRA+low anti-CD144 (concentration of 2×10 −6M antibody in solution).

FIG. 15. shows microscopy images of 6 well polystyrene plates with HUVEC cells subject to different conditions at 20 h of incubation. The surface conditions are the unmodified initial substrate (aminated polystyrene plate), RGD-TAMRA only, Anti-CD144 only (high), collagen, RGD-TAMRA+high anti-CD144, and RGD-TAMRA+low anti-CD144.

FIG. 16. shows ELISA results on L605 Cobalt-Chromium (CoCr) substrates modified with peptides (RGD-TAMRA:RRGW 50:50 mix) and antibodies (anti-CD309). (A) Without S-SMPB, peptides adsorb onto L605 which can change antibody orientation on surfaces. (B) 12-well plate with CoCr samples at the end of the ELISA. For custom ELISAs, RGD-TAMRA was detected using anti Rhodamine followed by HRP-labelled secondary antibodies. Anti CD309 was detected using an HRP-labelled F(ab′)2 secondary antibody on a separate set of samples. HRP activity was quantified by measuring A450 of supernatant collected after 30 min incubation with TMB substrate+addition of stop solution. Higher surface density of RGD-TAMRA (C) and anti-CD309 (D) were detected in the presence of S-SMPB. Peptide (E) and antibody (F) ELISA signal after 28d storage in the dark (ambient conditions) was significantly higher with covalent peptide grafting. The RGD-modified aminated CoCr surfaces significantly enhanced 3 h ECFC adhesion & spreading in static cultures compared to unmodified or aminated CoCr (G-I). *p<0.05; **p<0.01; ***p<0.001. N=3 L605 samples except G-I (N=9 images).

FIG. 17. shows that functionalized CoCr surfaces retain antibodies and RGD TAMRA peptides after gamma-irradiation as determined by enzyme-linked immunosorbent assays (ELISA). (A) Principle of the ELISA method used to detect immobilized antibodies via horseradish peroxidase (HRP) conjugated secondary antibodies. (B) ELISA method used to detect immobilized RGD-TAMRA peptides via primary anti rhodamine antibodies followed by HRP conjugated secondary antibodies. The means of ELISA signals for immobilized anti CD309 antibodies (C) and RGD TAMRA peptides (D) were practically equivalent (two one-side t-test; 15% difference considered to be practically equivalent).

FIG. 18 shows surface endothelialization of functionalized CoCr vascular stents. The combined effects of RGD peptides on cell spreading and capture antibodies on cell tethering lead to significantly increased endothelialization by ECFCs as measured by surface coverage on bi-functional surfaces compared RGD peptide-only or antibody-only controls. (A) Representative fluorescence images of ECFCs on the surface of modified stents (after plasma amination) and an unmodified control after 2 and half hours of circulating ECFCs over the stents in a perfusion model at 1 dyn/cm² wall shear stress. ECFCs were stained for F-actin (red) and nuclei (blue). Phase contrast images show the corresponding stent structure. Flow direction was from right to left. (B) Quantification of cell adhesion parameters from the fluorescence images. *P<0.05 and **P<0.01 with N=3 experimental replicas from different ECFC donors.

### DETAILED DESCRIPTION

It is provided a surface functionalized with cross linking groups, the surface adapted to receive antibodies and/or fragments thereof that bind a specific subtype of cells capturing the cells onto the functionalized surface and/or that bind cellular products, the surface comprising an antibody binding biomolecule comprising a linker region which is covalently crosslinked to functional groups on the surface and an antibody binding region that binds to any non-variable region of the antibodies or the fragments thereof and controls the orientation of the antibodies and/or the fragments thereof such that the variable regions are away from the surface, and the antibody binding biomolecule having a molecular weight of less than 10,000 g/mol; and a cell interacting biomolecule comprising a linker region which is covalently crosslinked to functional groups of the surface and a cell interacting region, the cell interacting biomolecule has a three dimensional structure such that the cell interacting region is oriented away from the surface when the linker region has crosslinked with the surface, and the cell interacting biomolecule has a molecular weight of less than 10,000 g/mol; wherein a concentration of the antibody binding biomolecule and a concentration of the cell interacting biomolecule on the surface are each independently controlled within a predetermined range that allows both the antibodies and/or the fragments thereof and the cell binding biomolecule to be functional.

The surface according to the present disclosure is functionalized to have a dual function by covalently binding two biomolecules on the surface. The first function of the surface is to capture a specific type of cell and/or a specific cellular product. This is achieved with an antibody-binding molecule which is conjugated on the surface and then further incubated with antibodies to allow their immobilization. By changing the immobilized antibodies on the surface, different cell types and/or different cellular products could be specifically targeted. Therefore, the surfaces as encompassed herein can recruit any cell type or product as long as there is a corresponding antibody or combination of antibodies to capture the desired group of cells or products. The second function is the adhesion, spreading, proliferation, differentiation, activation and/or other functional attributes of the cells on the surface which is achieved with a cell-interacting biomolecule that is covalently conjugated on the surface. The term “biomolecule” as used herein refers to molecules that are found in biological systems, that are derived from biological systems or molecules that are engineered/synthesized to be biocompatible and interact with biological systems. The biomolecule may comprise a peptide, a protein, a nucleic acid, a nucleic acid sequence, a lipid, a glycoprotein, a fluorophore, other biopolymers, or any combination thereof. The biomolecule may be a bioactive molecule such as synthetic polymers or synthetic molecules which interact with the cells.

Antibodies do not provide cells with the necessary signals to fully adhere, proliferate, or differentiate/function. Cell-interacting molecules such as ECM-derived peptides on the other hand lack the ability to capture cells under dynamic conditions due to slow adhesion kinetics and significantly smaller structure. Therefore, the combination of the two biomolecules in the present disclosure allows for an improved surface functionalization technique having the dual functions conferred by the antibody binding biomolecule and the antibody as well as the cell-interacting biomolecule. It is encompass that the antibodies described herein also can be seen as cell-interacting. For example antibodies can be used to block or activate cell receptors. Accordingly, antibodies and the smaller biomolecules could be used together to target distinct cell-activating pathways or mechanisms. Thus, the antibodies described herein can be used for cell capture and/or to activate or block a cell receptor, wherein the peptides can be used to trigger a secondary signal.

The surface according to the present disclosure can have any desired geometry such as a flat, concave, convex or complex geometry. For example, the surface may be a cell culture surface, the surface of a cardiovascular stent, the surface of microbeads or microcarriers. The surface can be an inert surface that is modified to have functional groups or a commercially available surface that has functional groups, which are covalently bound to the biomolecules. The functional groups may be amine, carboxylic acid, thiol, carbonyl or any other groups suitable for the formation of the covalent bond with the biomolecules. The functional groups may be present in the underlying surface or introduced through surface modification (e.g. chemical or plasma treatment of the surfaces; adsorption of polymers or other molecules containing these functional groups). In some embodiments, the surface modifications are applied to polymers or metals (e.g. CoCr). In some embodiments, prior introduction of functional groups on the substrate for example by coating (e.g. with polymers that contain nitrogen or other functional groups such as polylysine or polydopamine), chemical modification (e.g. silanization) or plasma treatments (as we have done on CoCr) are needed.

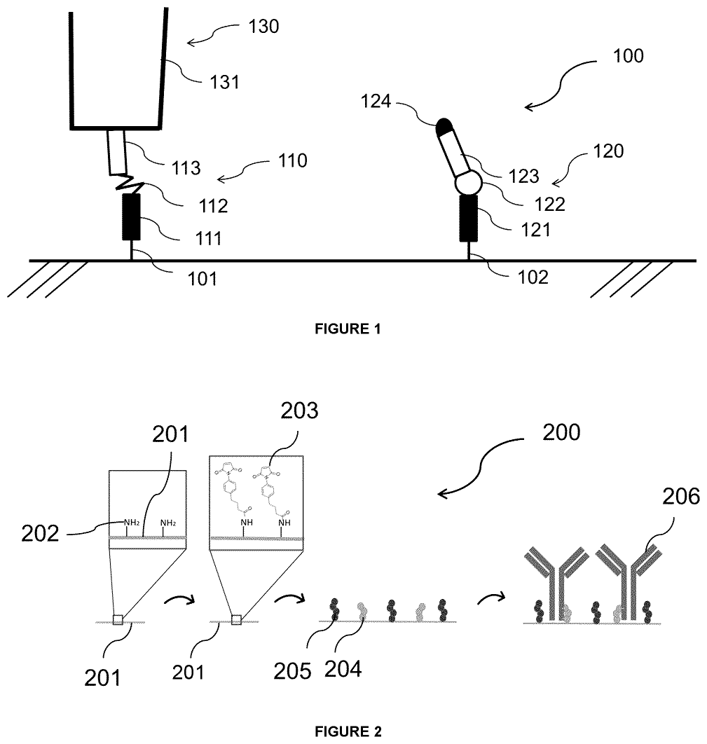

In some embodiments, the surface is used for cellular assays. For example, the antibodies immobilized on the surface can be specific to cellular products that are desired to be quantified. Cellular products include secreted molecules such as hormones and cytokines or any signaling molecule. Other examples include yeast or bacterial cells that have been modified to secrete a specific product. In certain embodiments, antibodies can be used for cell capture and other antibodies can be further immobilized for quantification of their cellular products. Cellular products are not necessarily secreted by the cells as they can be released in the medium after cell lysis or permeabilization of cell membranes. The quantification analysis of the cellular products binding to the antibodies can be done by enzyme-linked immunosorbent assay (ELISA), enzyme-linked immunospot (ELISPOT), western blot or any other suitable means depending on the assay. The cellular assays include cell culture assays, and in one example, the antibodies target cytokines to reduce the amount of harmful cytokines in the medium, or to “track” what the cells are secreting. In this case, the molecules secreted could be quantified at the end of the culture by adding a secondary antibody and detection using conventional sandwich ELISA assays—similar to ELISPOT assays. Making reference to FIG. 1, the surface 100 according to the present disclosure has an antibody binding biomolecule 110. The antibody binding biomolecule 110 has a linker region 111 that is bonded to the surface by a covalent bond or crosslink 101. In one example, the linker region 111 has linking arm that is a bilinker or a trilinker. In some embodiments, the linker region can comprise, and not limited to, a crosslinker that is a maleamide crosslinker, a carbodiimide crosslinker, an imidoester crosslinker or a N-Hydroxysuccinimide Ester (NHS Esters) crosslinker. For example, the crosslinker can be selected from the group consisting of disuccinimidyl substrate (DSS), sulfosuccinimidyl 4-(n-maleimidophenyl)butyrate (Sulfo-SMPB), sulfosuccinimidyl 4-(n-maleimidophenyl)cyclohexane-1-carboxylate (Sulfo-SMCC), 1-Ethyl-3-(3-dimethylaminopropyl)carbodiimide (EDC), Sulfo-NHS and N-κ-maleimidoundecanoyl-oxysulfosuccinimide ester (Sulfo-KMUS). Depending on the available functional groups on the surface, the linker region might consist of one or more of these crosslinking arms.

The antibody binding biomolecule has an antibody binding region 113 comprising an amino acid sequence or a nucleic acid sequence that binds to any non-variable region of antibodies or antibody fragments. In the embodiment exemplified in FIG. 1, the antibody binding biomolecule binds to the fragment crystallizable region (Fc) 131 of the antibody 130. The amino acid sequence can be a peptide or a small protein that binds the Fc region 131 as long as the antibody binding biomolecule has a total molecular weight smaller than about 10,000 g/mol, about 9,000 g/mol, about 8,000 g/mol, about 7,000 g/mol, about 6,000 g/mol, about 5,000 g/mol, about 4,000 g/mol, about 3,000 g/mol, about 2,000 g/mol or about 1500 g/mol. In some embodiments, the molecular weight of the antibody binding biomolecule is between about 500 g/mol to about 2000 g/mol, between about 650 g/mol to about 1750 g/mol, or between about 800 g/mol and about 1500 g/mol. Examples of such peptides or small proteins include RRGW, HWRGWV, CHKRSFWADNC, CPSTHWK, NVQYFAV, ASHTQKS, QPQMSHM, TNIESLK, NCHKCWN, SHLSKNF, NKFRGKYK, NARKFYKG. The electrostatic interactions near hydrophobic patches are a good binding site target because of the entropy gained from repelling bound water around the hydrophobic patch. In one embodiment, the amino acid sequence is free of any bacterial peptides, proteins and derivatives thereof. The antibody binding region 113 is positioned relative to the linker region 111 and the surface 100 such that the orientation of the variable regions of the antibodies are away from the surface. In one embodiment, oriented away from the surface is defined as being at a distance that reduces the steric hindrance of the surface. In one embodiment, the antibodies are non-natural antibodies created for example via chimerism. In that case, the two hypervariable regions may bind different antigens for example. In certain embodiments, the antibodies or fragments thereof are immobilized by glycosylation to the antibody binding biomolecule.

The antibody binding biomolecule optionally has one or more spacing regions 112 to improve the performance of the antibody binding biomolecule. The spacing regions can be used to reduce the steric hindrance induced by the surface and/or to minimize non-desirable and non-specific protein adsorption on the surface when in contacts with fluids containing other proteins such as blood, plasma or cell culture medium. The spacing regions can comprises polyethylene glycol, amino acids (such as G and C), propylene sulfoxide, and/or other compounds with similar polar functional groups, net charge, hydrogen bond acceptor groups, and hydrogen bond donor groups.

Most of the antibody surface immobilization techniques available for in vitro studies or for in vivo cell capture applications according to the prior art rely on direct adsorption, surface conjugation via primary amines, or interactions with bio-affinity bacterial proteins. The simplest method to immobilize antibodies on surfaces is adsorption, but this method can lead to a reduction in antigen binding due to desorption and conformations with reduced availability of antigen-binding sites. Although antibody conjugation via primary amines, carboxylic acids or other functional groups on antibodies is less susceptive to desorption, this technique suffers from a lack of control over antibody orientation on the surface due to the prevalence of primary amine throughout the antibody structure. Directional antibody immobilization can be achieved via binding of the Fc region to surfaces grafted with bacterial products such as protein A or G. Disadvantages of this strategy include the immunogenicity of these bacterial proteins and their high affinity for albumin, which may increase fouling in the presence of biological fluids. Furthermore, protein A and G have a large molecular weight of more than 30,000 g/mol and more than 20,000 g/mol respectively which could explain why these two proteins are not suitable for the present dual function surface.

Due to the disadvantages and limitations of such immobilization methods it is desirable to use easily synthesized peptides or nucleic acids, such as for example aptamers, that are able to both immobilize antibodies and control their orientation. For instance, the short peptide sequence RRGW has a strong association toward the Fc region of mouse immunoglobulin G (lgG). With this and other short synthesized peptides, it becomes convenient to screen a large number of antibodies in a single experiment through an easy switch between different antibodies. The additional advantage of using these peptides to immobilize antibodies rather than proteins, such as protein A or G, is their small size that provides better control in grafting the molecule on the surface and minimizing steric hindrance.

The cell interacting biomolecule 120 has a linker region 121 that is bonded to the surface by a covalent bond or crosslink 102. The linker region 121 comprises a crosslinker that can be a maleimide crosslinker, a carbodiimide crosslinker, an imidoester crosslinker or a N-Hydroxysuccinimide Ester (NHS Esters) crosslinker. For example, the crosslinker can be selected from the group consisting of disuccinimidyl substrate (DSS), sulfosuccinimidyl 4-(n-maleimidophenyl)butyrate (Sulfo-SMPB), sulfosuccinimidyl 4-(n-maleimidophenyl)cyclohexane-1-carboxylate (Sulfo-SMCC), 1-Ethyl-3-(3-dimethylaminopropyl)carbodiimide (EDC), Sulfo-NHS and N-κ-maleimidoundecanoyl-oxysulfosuccinimide ester (Sulfo-KMUS). Depending on the available functional groups on the surface, the linker region might consist of one or more of these crosslinking arm. Thus, for example the linker region has a bilinker or a trilinker arm.

The cell-interacting biomolecule 120 has a cell-interacting region 123 that interacts with the cellular surface molecules such as cell surface receptors. The interaction may be direct, such as integrin binding to ligands on the surface, and/or indirect such as receptor binding with ligands that trigger receptor activation and downstream signaling within the cell. Examples of such cell surface receptors are integrins, growth factor receptors, other signaling proteins, as well as other components present in the cell membrane or on its surface which can relay signals to the cells. In one embodiment, the cell-interacting region comprises a ligand that can be a peptide or small protein derived from extracellular matrix (ECM) proteins, growth factors, hormones, other proteins which bind cell-surface receptors or molecules engineered to bind to these receptors such as an engineered aptamer. Surfaces modified with ECM-derived peptides promote cell adhesion, proliferation, and differentiation making it ideal for tissue engineering scaffolds and cell expansion platforms. The ligand can be derived from common motifs that contribute to the cell adhesive properties of various ECM proteins including fibronectin, vitronectin, fibrinogen, osteopontin, and some collagens. These motifs may be between 3 to 100 or even between 3 to 20 amino acids. For example, RGD is a common motif that can be used as ligand. Other examples include but are not limited to REDV, DEGA, YIGSR, RNIPPFEGCIWN, PHSRN, KRSR GFOGER, GPEILDVPST and IKVAV. The ligand can be derived from common motifs that promote cell survival and proliferation present in ECM proteins or growth factors such as vascular endothelial growth factor, basic fibroblast growth factor (bFGF), epidermal growth factor (EGF), insulin, insulin-like growth factor or others. The ligand can be derived from motifs that contribute to cell differentiation or function such as cell signaling proteins or hormones. Examples of these ligands include hormone peptides such as adrenocorticotropic peptide (ACTH-(4-10), MEHFRWG-OH), growth factor-derived peptides such as FREG peptide (DPHIKLQLQAE) and cell migration-mediated peptides such as SRSRY peptide. When cells are captured on the surface, the cell interacting region enters into contact with molecules at the cell surface which promotes the adhesion, proliferation, spreading, differentiation and/or functional response of the cells on the surface. The cell interacting biomolecule has a three dimensional structure such that the cell interacting region is oriented away from the surface when the linker region has crosslinked with the surface. This reduces the steric hindrance exerted by the surface. “Oriented away from the surface” can be defined as being at a distance from the surface of at least 8, 9, or 10 angstrom. This is achieved by reacting the biomolecules with the linker that was introduced on the surface through their terminal crosslinking group, thus controlling the biomolecule's orientation. In some embodiments, the cell interacting region comprises a peptide at the end tail of the biomolecule.

The cell interacting biomolecule has a total molecular weight smaller than about 10,000 g/mol, about 9,000 g/mol, about 8,000 g/mol, about 7,000 g/mol, about 6,000 g/mol, about 5,000 g/mol, about 4,000 g/mol, about 3,000 g/mol, about 2,000 g/mol or about 1500 g/mol. In some embodiments, the molecular weight of the antibody binding protein is between about 500 g/mol to about 2000 g/mol, between about 650 g/mol to about 1750 g/mol, or between about 800 g/mol and about 1500 g/mol.

The cell interacting biomolecule optionally has one or more spacing regions 122 to improve the performance of the antibody binding biomolecule. The spacing regions can be used to reduce the steric hindrance induced by the surface and/or to minimize non-desirable and non-specific protein adsorption on the surface when in contacts with protein-containing fluids. The spacing regions can comprises polyethylene glycol, amino acids (such as G and C), propylene sulfoxide, and/or other compounds with similar polar functional groups, net charge, hydrogen bond acceptor groups, and hydrogen bond donor groups. The spacing may be a capping end 124 consisting of one or more amino acids.

Surface modification strategies that can combine a small biomolecule (less than 10,000 g/mol) and antibodies are scarce and non-controllable due to the vast difference in their molecular weight. For example, a peptide can be over 150 times smaller than antibodies, therefore it can easily bind to the structure of the antibody itself and not the surface if they are simply mixed in solution. Reported methods mainly consist of conjugations of different growth factors or morphogenesis proteins independently or using a simple mixture of those molecules with no control over overall resulting surface density or the orientation of the biomolecules and antibodies. The techniques known today have been limited to the immobilizing of either antibodies alone or ECM-derived molecules alone as each of these biomolecules has a unique function. Some of these known technologies also rely on adsorption forces to modify the surface with the biomolecules of interest which is easily washed away upon exposure to aqueous conditions such as cell culture media and bodily fluids. Thus previously known techniques lack control over the resulting surface concentration of the grafted molecules which hinders the success of the technology due to regulatory constraints. They also mostly lack the ability to control the orientation of both biomolecules which reduces the biological effect of the surface.

The above mentioned problems are overcome by the surface as proposed herein and according to the present disclosure. The cell interacting biomolecule and the antibody binding biomolecule crosslinked to the surface have similar sizes. Furthermore, as previously described, the biomolecules are covalently bound to the surface with a controlled orientation and the antibodies are also immobilized with a controlled orientation due to the presence of the conjugated antibody binding biomolecule. During surface functionalization, applying molecules similar in size avoids interactions in solution which may impact the final surface density of the antibodies and cell-interacting biomolecules. The molecular weight ratio between the cell interacting biomolecule and the antibody binding biomolecule is between about 2:1 to about 1:2, about 1:3 to about 3:1, about 1:4 to about 4:1, about 5:1 to about 1:5, or between about 1:10 to about 10:1. Furthermore, because the biomolecules are conjugated to the surface, the resulting surface concentration of the biomolecules and antibodies can be tightly controlled by changing the concentration applied to the surface. The combination of the small size of the biomolecules, the control of the orientation of the molecules, the similarity in size between the biomolecules, and their respective function yields an advantageous surface that can be used for cell capture and retention across most of the surface. Applications for such a surface include but are not limited to dynamic cell culture, a mimic of biological cell interfaces or walls, vascular stents, engineered grafts and microcarrier applications.

The cell-interacting biomolecules and the antibodies can be applied as mixtures. For example, mixtures of cell-interacting biomolecules can be applied to confer different functional attributes such as cell adhesion and cell differentiation. To achieve this, cell-interacting biomolecules can be mixed in different ratios with antibody-binding peptides prior to antibody immobilization. Similarly, mixtures of antibodies can be applied to optimally capture one cell type or cellular product which expresses corresponding antigens, or to capture more than one desired cell type. This can be achieved by mixing antibodies in different ratios during the antibody immobilization step. As an example, after grafting the linking arm to surfaces, a mixture of RRGW peptide, RGD-TAMRA peptide and a growth factor-derived peptide motif can be mixed and conjugated via free thiol groups. Due to the similar size of these peptides, the surface density can be adjusted by changing the concentration of these peptides in solution during covalent grafting. Next, a mixture of antibodies such as CD31 and CD309 can be applied in solution to immobilize both antibodies in the same step. By conjugating the cell-interacting biomolecules and the antibody binding biomolecule prior to immobilizing the antibodies, several cell-interacting biomolecules and several antibodies can be applied while retaining control over the final surface density of each component. No additional reaction steps are required to add more than one cell-interacting biomolecule or more than one antibody on the surface aside from adjusting concentrations in solution during surface modification steps.

The surfaces of the present disclosure are prepared with the following method. Making reference to FIG. 2 showing the method 200 of producing the surface according to one embodiment. First, a surface 201 is provided with a functional group 202 such as an amine as shown in FIG. 2. The functional group 202 may be any suitable group that will crosslink with a linker 203 such as amine, thiol, carboxyl or carbonyl. Alternatively, an inert surface can be provided and subsequently activated by chemical treatment, plasma treatment or other suitable methods to modify its surface to have any functional groups 202.

Second, the linker 203 is reacted with the functional groups to form a covalent bond as shown in FIG. 2 with the example of Sulfo-SMPB linker arm 203 binding the amine group 202 covalently. The linker can be a maleimide crosslinker, a carbodiimide crosslinker, an imidoester crosslinker or a N-Hydroxysuccinimide Ester (NHS Esters) crosslinker. The linker 203 shown in FIG. 2 is a Sulfo-SMPB but any suitable linker can be used. For example the linker is selected from the group consisting of DSS, Sulfo-SMPB, Sulfo-SMCC, EDC, and N,N′-Dicyclohexylcarbodiimide. The surface 201 can be washed to remove the linkers that have not linked with a crosslinking group 202.

Then, an antibody binding biomolecule 204 is crosslinked to the linker 203 at the linker region of the antibody binding biomolecule by adding a predetermined concentration of the antibody binding biomolecule 204 onto the surface 201 thereby forming the complete biomolecule. Due to the antibody binding biomolecule covalently linking with the surface, the concentration of the immobilized antibodies on the surface 201 can be controlled by the concentration of antibody binding biomolecule in the mixture prior to application on the surface. The antibody binding biomolecule 204 has an antibody binding region that binds to the Fc region of antibodies and controls the orientation of the antibodies such that the variable regions are away from the surface, and the antibody binding biomolecule has a molecular weight of less than 10,000 g/mol.

Further, substantially in conjunction or substantially simultaneously, a cell interacting biomolecule or mixture of cell-interacting biomolecules 205 is crosslinked to the linker 203 at the linker region by adding a predetermined concentration of the cell interacting biomolecule 205 onto the surface 201 thereby forming the complete biomolecule. The cell interacting biomolecule 205 has a cell interacting region that interacts with cellular receptors of the cells to promote the adhesion, spreading and/or proliferation of the cells on the surface 201. The cell interacting biomolecule has a three dimensional structure such that the cell interacting region is oriented away from the surface when the linker region has crosslinked with the surface, and a molecular weight of less than 10,000 g/mol. The surface may then be washed to remove the biomolecules that have not reacted to the linker groups.

Finally, antibodies 206 are incubated on the surface and allowed to bind to the antibody binding biomolecule 204. Depending on the application of the surface, a secondary antibody is used to detect the presence of the first antibody. Because the antibody or mixture of antibodies is not immobilized directly onto the surface 201 but is linked to the surface through a previously deposited antibody binding biomolecule, the orientation of the antibodies can be controlled. The concentration of antibodies is tightly controlled by changing the concentration of the antibody binding biomolecule 204. Contrary to applying antibody binding biomolecule, the direct conjugation of antibodies on the surface via amine or carboxylic acid functional groups reduce the control over their orientation. Due to the prevalence of such groups throughout the antibody structure, the Fab regions—necessary for cell capture—might be immobilized on the surface leading to diminished-activity of the antibody.

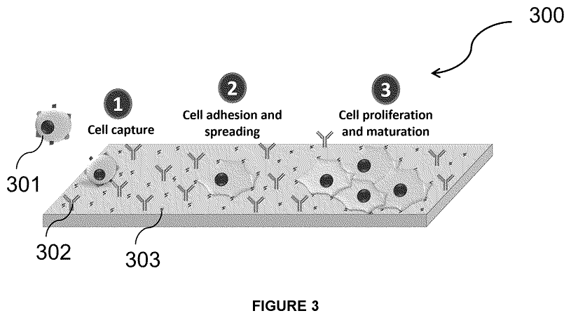

The dual function obtained for the surface of the present disclosure is shown in FIG. 3. A surface 300 recruits cells 301 with the antibodies 302 being properly oriented to maximize the capture of cells 301. The recruited cells 301 then interact with the cell interacting biomolecule 303 which promotes the adhesion, spreading, and/or proliferation.

The following example uses a RRGW biomolecule and a RGD biomolecule however the concentrations described are in no means limiting. In fact, the concentrations in the present method can be optimized for different antibody binding biomolecules and cell interacting biomolecules as performed in the example below for the combination of RRGW biomolecule and RGD biomolecule.

### Example I

Surface Functionalized with RGD Biomolecules and RRGW Biomolecules

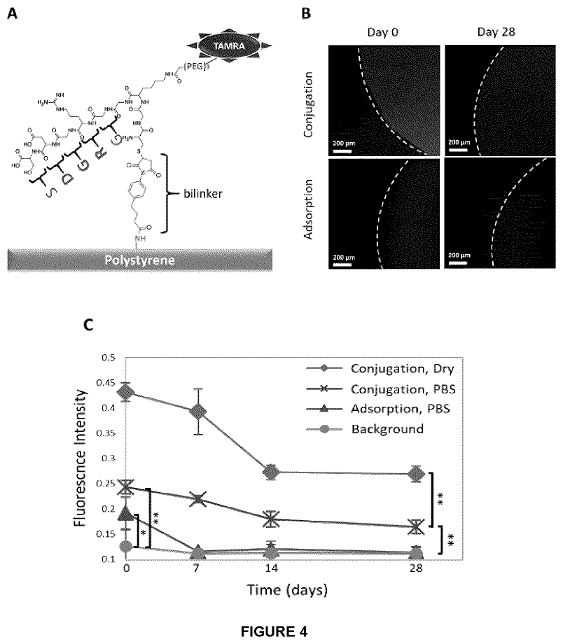

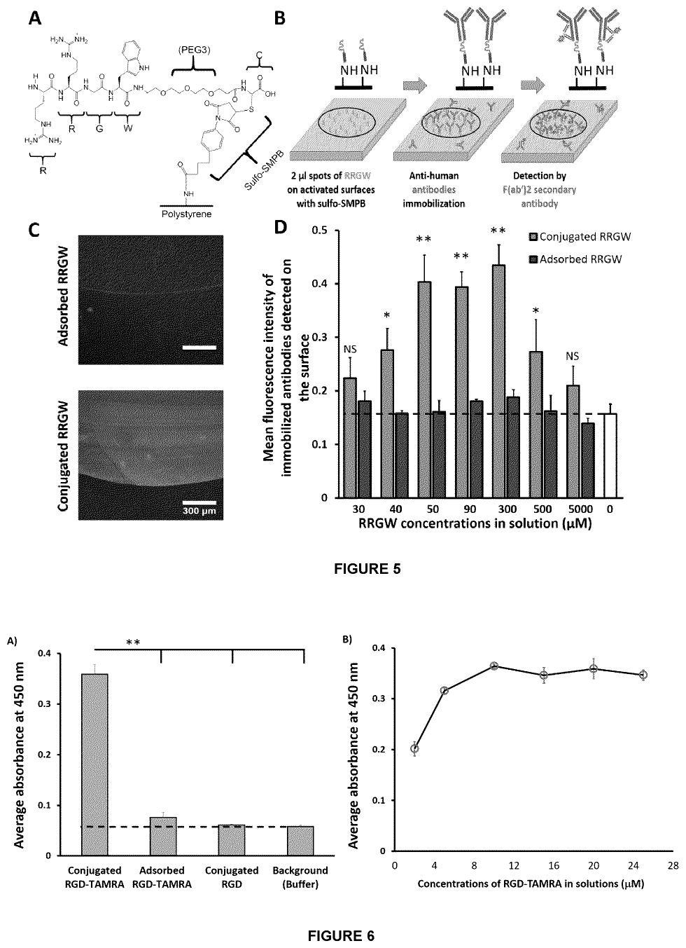

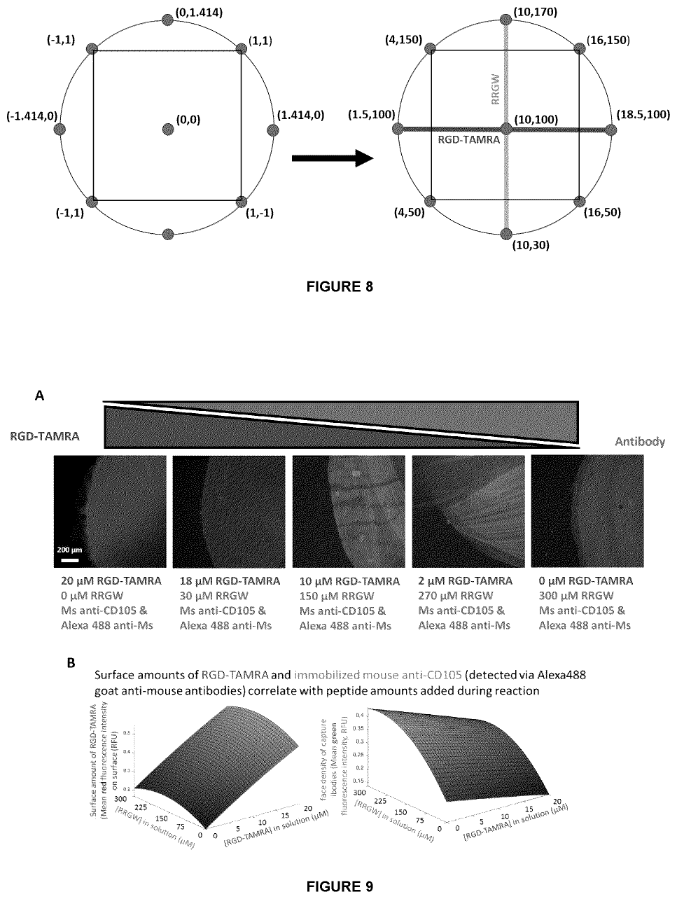

To form the surface, the first step was to graft Sulfo-SMPB— a heterobifunctional linker—to activate an aminated surface and to introduce free maleimide groups available for reactions. To have a better control over the dual conjugation strategy, two peptides that are relatively equal in size and have terminal thiol groups were synthesized to have similar chemical reaction kinetics with the surface. The two synthesized peptides, antibody-binding peptide [RRGW(PEG3)C] (FIG. 4) and ECM-derived peptide [CGKGGRGDS(PEG3)-TAMRA] (FIG. 5), were reacted with the available maleimides that were introduced on the surface from the first step through their terminal thiol, thus controlling the peptide orientation. Two-microliter spots of a mixture of both peptides with different ratios and concentrations were created to analyze the surface modification via a response surface model. Then, an immunoglobulin G capture antibody was added to cover the surface followed by a secondary antibody (F(ab′)2) conjugated with Alexa Fluor 488-, a green fluorophore molecule. A confocal microscope was used to detect the intensity of the antibody via a green channel and the ECM-peptide via a red channel with the presence of TAMRA, a red fluorophore molecule. A central composite circumscribed (CCC) was used to identify the independent and interdependent effects of the concentration of both molecules on the signals of the two wavelengths, Red and Green, which correlates to the surface concentrations of each. A schematic representation of the CCC design is shown in FIG. 8: the normalized values of each point in the model, and the concentrations in solutions (in NM) of RGD-TAMRA and RRGW in X-axis and Y-axis, respectively.

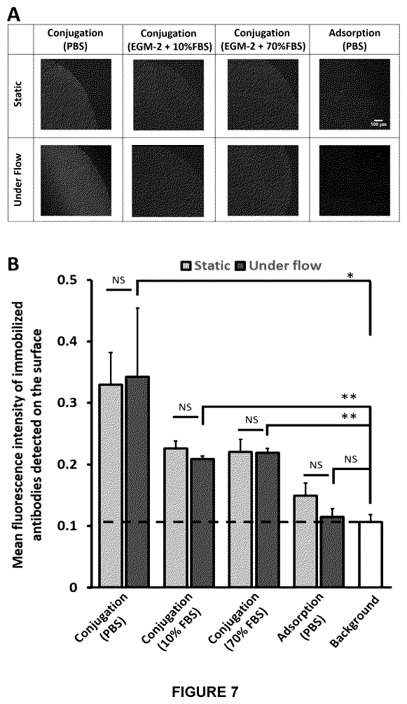

The CCC experimental design was used to build surface responses model with two factors, the first one was the concentration of the RGD-TAMRA peptide in solution, and the second one was the concentration of the RRGW peptide in solution. The outputs of the model were the fluorescence intensity of RGD-TAMRA and the fluorescence intensity of the secondary antibodies. This model contains a 2 by 2 factorial design, a central point and 4-axial points that test the extremes conditions of each factor. The RGD-TAMRA (FIG. 4A) and RRGW (FIG. 5A) peptides were both synthesized to be between 800-1500 g/mol with a terminal thiol group for surface grafting and with enough spacing from the surface to avoid hindrance. The surface conjugation of each of the peptides alone was studied by using different initial peptide concentrations to identify a range of concentrations where the peptide can react with the surface without saturating the available functional groups (FIGS. 4 & 5). The durability of the surface conjugation of each of the peptides under different conditions was also confirmed (FIGS. 6, 7 and FIG. 17). Mixtures of RGD-TAMRA and RRGW peptides were then prepared in the identified ranges of concentrations.

Spots of 2 μL (around 0.64 μL/mm²) of each mixture were added to be conjugated on aminated surfaces activated with sulfo-SMPB and incubated statically for two and a half hours in the dark, and then the slides were rinsed twice with PBS. Slides were then blocked by Dako serum-free protein block, followed by covering the entire surface with: first 10 pg/mL mouse anti-CD144 and second by 10 pg/mL F(ab′)2-goat anti-mouse IgG conjugated with Alexa Fluor 488. Slides were lastly washed twice with PBS and RO water, respectively. The spots were then imaged using the same confocal microscope using a 10× objective. Every spot was imaged twice, once at a green wavelength to detect the immobilized antibodies, and second at a red wavelength to detect the RGD-TAMRA. For each condition, 3 spots with 6 images for each spot were analyzed. The central point was triplicated to increase the confidence level and decrease the error in the model.

For the results of the bifunctional surface modification experiment, the effect of two independent variables (the concentrations of both peptides, RGD-TAMRA and RRGW, in solution) on two response variables (the TAMRA intensity and the secondary antibodies intensity) was determined using the fit model option in JMP®, a statistical software. Independently for each response, the lack of fit was verified and considered significant at P<0.05. For each measured output, only parameters or factors that have p<0.05 were considered to have a significant effect.

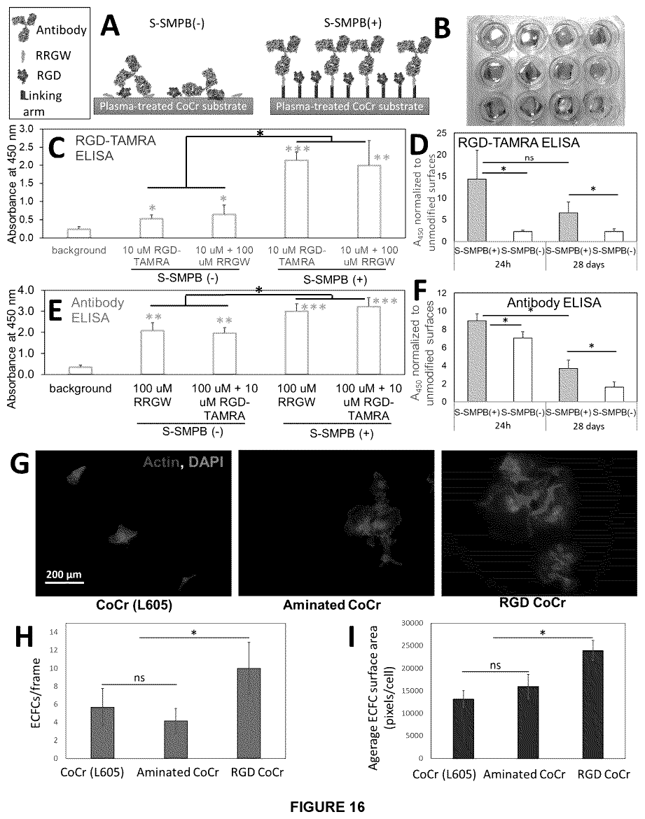

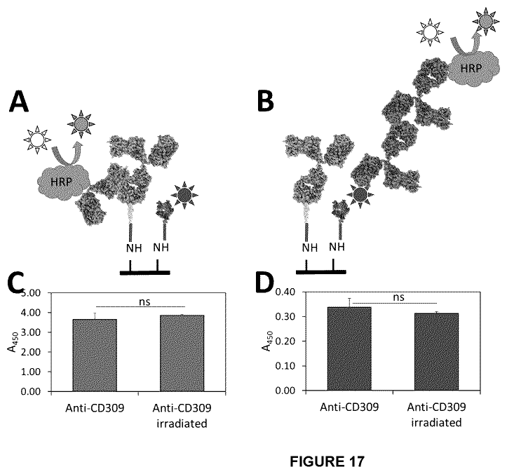

As described above, to combine both antibodies and ECM-derived peptides, different concentrations of both RRGW and RGD-TAMRA were mixed and spotted on the surface. The CCC design was built to assess the effect of both peptides in the final responses. These surface responses were predicted by measuring the fluorescence intensities of both TAMRA and Alexa Fluor 488, which are conjugated to RGD and the secondary anti-mouse antibodies, respectively (FIG. 9A). Even though an ELISA was developed previously to detect the RGD-TAMRA on the surface (FIG. 6, FIG. 16 & FIG. 17) and to study its saturation curve, it was easier and less-material consuming to use fluorescence microscopy in this study to build the model. While the same 2 μL spots were used to detect the two outputs (surface responses) in this study, at least 100 μL of every prepared solution would have been required to fill one well in the 96-well plate with the need of applying two different detection methods (one ELISA for RGD-TAMRA and another one for antibodies).

Interestingly, no significant interaction occurred between the two peptides on the measured surface responses. This model strongly suggests that both peptides could be conjugated on the surface in a very controllable manner. FIG. 9B shows the predicted surface responses regarding the different concentrations of both peptides, while FIG. 9A shows different representative fluorescence imaging of various ratios of both molecules spotted on the surface.

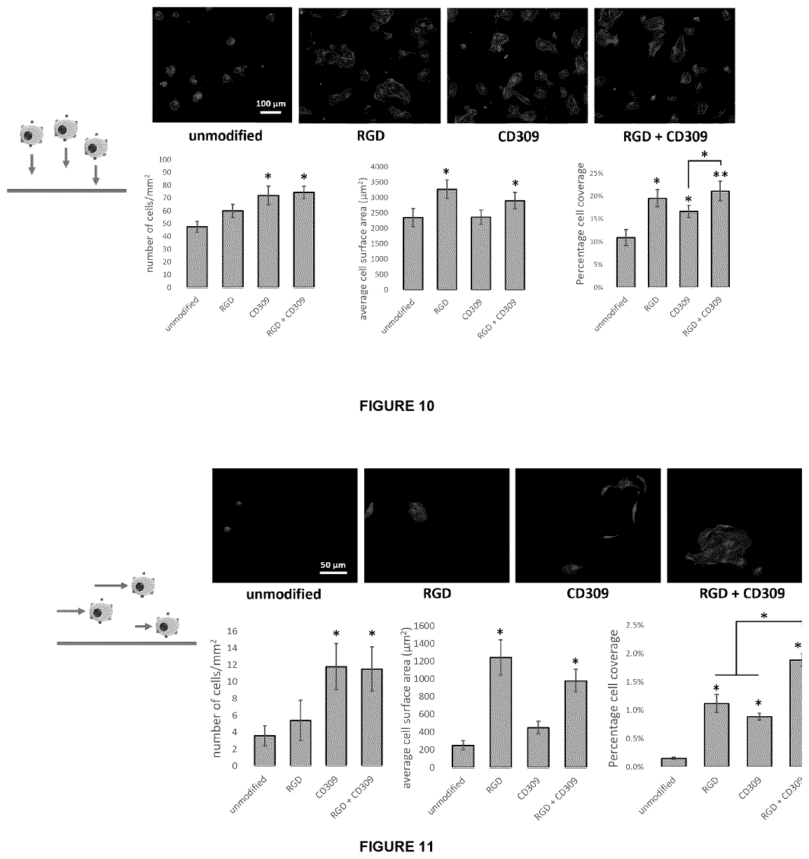

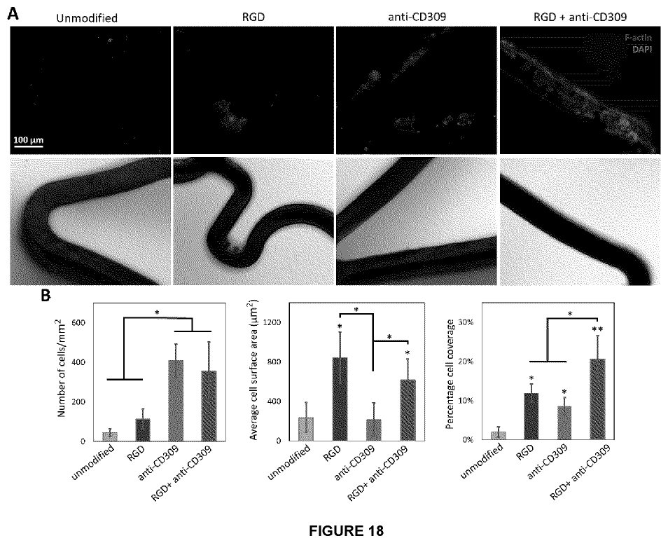

By applying the present method with commercially available aminated polystyrene surfaces, the behavior of endothelial colony forming cells (ECFCs) towards the modified bifunctional surfaces was tested in static conditions (FIG. 10) and under flow (FIG. 11). Surprisingly it was found that modified surfaces have higher ECFC surface coverage, which represents the product of the number of captured cells and their spreading, compared with surfaces with either antibodies alone or the ECM-derived peptide alone (FIG. 11). The same effects were observed on flat polystyrene (FIG. 11) and CoCr substrates, as well as CoCr stents (FIG. 18). For CoCr, amine groups were introduced via plasma treatment using known methods.

To test whether the bifunctional surfaces can impart two significant effects on cells of interests, ECFCs derived from human peripheral blood were either seeded on the modified surfaces under static conditions or flown over the surfaces in a perfusion loop under 1 dyn/cm² wall shear stress. The RGD-TAMRA peptide and anti-CD309 antibodies immobilized using the RRGW peptide were selected due to each of their demonstrated positive effects on ECFC spreading and ECFC capture respectively. The two molecules were mixed together at the center point concentrations of the previously described surface model. Under static conditions, the number of ECFCs on antibody surfaces and bifunctional surfaces were significantly higher than the controls (unmodified surfaces) due to the presence of the cell capturing effect associated with the antibody. On the other hand, the surface area of the ECFCs on the RGD-TAMRA and the bifunctional surfaces were significantly higher than the controls due to the presence of the cell spreading effects associated with the peptide. Combining these two pieces of data, a maximum cell coverage is seen (FIG. 10) on the bifunctional surfaces which is a favorable outcome for medical devices and cell culture platform applications.

Under dynamic flow conditions this effect was even more evident as shown in FIG. 11 and FIG. 18. The cell adhesion data highlights two distinct cellular response processes: cell capture and cell spreading. These two processes are combined in the bifunctional surfaces and lead to the doubling of the cell surface coverage. The prominence of this effect under dynamic flow conditions is particularly important because it mimics the conditions on the surface of a blood-contacting medical devices which often have a dynamic interface with significant levels of wall shear stress due to blood flow. Suspension cell culture systems such as microcarriers are also usually in contact with the cells under dynamic stirring conditions.

As described herein, surfaces were modified by a combination of two molecules, where each one had a unique function. Combining antibodies with peptides on the surface with control over surface concentrations and orientations has proved to confer desirable advantages to the surface. Without wishing to be bound by theory, it is believed that the antibodies would increase the selectivity of capturing circulating cells, while RGD— the peptide—would promote the adhesion and proliferation of the captured cells. To achieve this, two peptides, RRGW and RGD-TAMRA, were combined in different concentrations and conjugated on the PureCoat′ aminated polystyrene surfaces, followed by the addition of primary antibodies. Combining these relatively similar-sized peptides at the chosen range of concentrations increased the opportunity of having a controlled method as neither of these peptides were expected to interact with each other or block the surface. This was confirmed by the central composite design, where no significant interactions were observed between the two conjugated peptides on the surface. This model was also used to predict the surface responses that result from the combination of both molecules on the surface (FIG. 9).

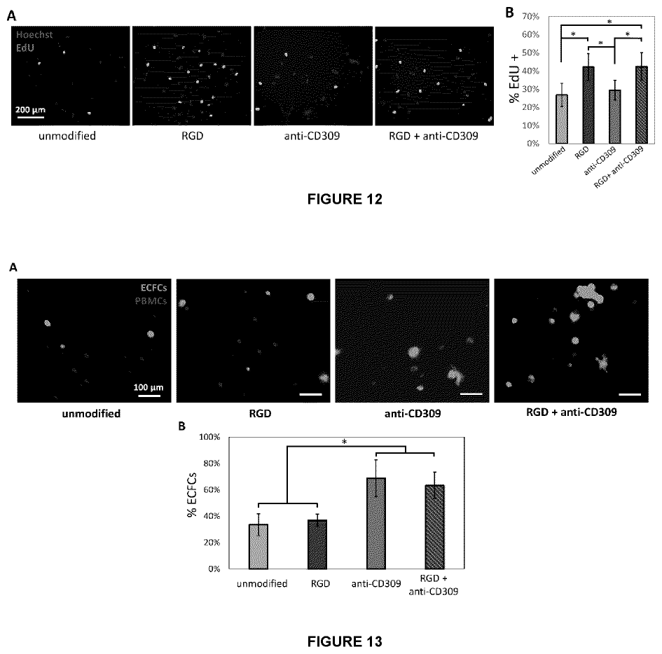

The cell adhesion experiments demonstrated the significant effect of the bifunctional method on improving the cell coverage on the surface (number of cells and their spreading) when compared to antibodies or peptides alone. This technique is useful in blood contacting medical devices for the aim of accelerating reendothelialization by capturing and recruiting EPCs on the surface which reduce biocompatibility issues. It is also very valuable for the modification of cell culture systems as it enables both selection (FIG. 13) and cell growth (FIG. 12) on the same platform, thus, reducing the number of required process units. It can also be used to modify tissue engineering scaffolds to recruit different cell types and create functional multicellular tissues.

Results showed significant trends resulting from varying the concentration of each of two peptides in their respective detected signals and no significant effects of either of the molecules on the other's signal response, suggesting that the method is very controlled with no significant interactions between the two molecules. Therefore, it is possible to choose the desired response by simply choosing the concentrations of each of the molecules in the mixture from an identified range of operation.

As encompassed herein, the modifications can be applied to different aminated substrates—as shown on polystyrene (FIGS. 4-13) and on cobalt-chromium (CoCr) (FIGS. 16-18). The modifications resist to sterilization methods such as gamma rays as shown in FIG. 17. The modifications can be applied to different geometries while retaining the benefits observed on flat substrates. On CoCr stents, ECFCs were captured from flow through antibodies and showed higher cell spreading in the presence of RGD. Due to the combination of these effects, bi-functional stents with both antibodies and RGD led to the highest cell surface coverage after 2 h of flow culture as shown in FIG. 18.

Also encompassed are variations to this exemplary method. Indeed, the method offers flexibility for applications on different material surfaces with different functional groups (i.e. not only primary amines) by simply using a different linker. It can also be used to immobilize any peptides with a terminal thiol group and any IgG antibody which makes it versatile for screening purposes and useful for various applications. The same principle can also be used to immobilize biomolecules other than peptides and antibodies. For example, growth factors and full native proteins can be combined with antibodies or peptides using the same strategy. The present technology allows for the creation of surfaces that can better mimic the complex multi-functional processes that are involved in cell recruitment in vivo.

### Example II

Unsuccessful Surface Functionalization with Protein G

The CD144 or the vascular endothelial cadherin (VE-Cadherin) antigen is a 140 kD glycoprotein. It is a calcium-dependent transmembrane cell-cell adhesion molecule localized at the intercellular boundaries of endothelial cells, hematopoietic stem cells, and perineurial cells. CD144 is thought to play a role in vascular development, permeability, and remodeling.

A 6 well plate was seeded with the following conditions in each well (W1-W6):

| | | |
|---|---|---|
| W1 - Purecoat amine | W2 - RGD-TAMRA 2 × 10⁻⁵M | W3 - Protein G 5.5 × 10⁻⁶M |
| | | 1/100 anti-DC144 VE-cadherin |
| W4 - Collagen | W5 -Potein G 5.5 × 10⁻⁶M (High) | W6 - Protein G 2 × 10⁻⁶M (low) |
| | 1/100 anti-CD144 VE-cadherin | 1/100 anti-CD144 VE-cadherin |
| | RGD-TAMRA 2 × 10⁻⁵M | RGD-TAMRA 2 × 10⁻⁵M |

The functionalized surface was rinsed with phosphate buffer saline (PBS) and reactive oxide water. HUVEC cells were used and were seeded in each well in serum free medium at a concentration of 19,000 cells/mL. The plate was incubated for 3 hours on an incubation shaker then imaged under the microscope. The plate were left to incubate for another 21 h (total 24 h) at 37° C. 5% CO₂ and imaged again.

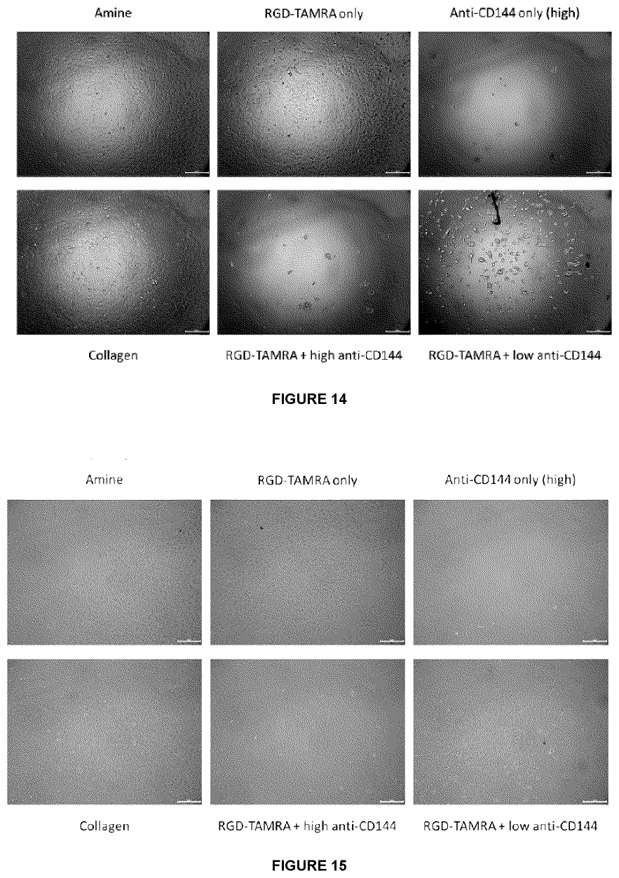

Results at 3 h are shown in FIG. 14. The cells can mostly be seen in the middle of the well and strongly attach in amine, collagen, RGD only condition (well 1-2 and 4). Only a few cells are attached to the surface when CD144 is used. Cells seem to spread more on the collagen surface and there are more cells in the well that has low protein G concentration.

Results at 20 h are shown in FIG. 15. The cells can be seen mostly in the middle of the well and are strongly attached in each condition. However, the wells with high RGD-TAMRA and high anti-CD144, and high-CD144 only have a significantly lower number of cells adhered and spread on the surface compared to all other conditions. It seems RGD-TAMRA and CD-144 (immobilized with protein G) may be counter acting the effect of each other which renders the surface performance worse than the control of Amine. Without wishing to be bound by theory, it is possible that the difference in size between RGD-TAMRA and protein G (which was used to conjugate the antibody) plays a role in the counter action observed. One explanation of this might be due to the localization of the peptide within the protein G molecule leading to hinder the interaction between the cell receptors and the peptide, which might be explained by comparing the condition of RGD-TAMRA alone with the condition of RGD-TAMRA combined with low anti-CD144.

While the present disclosure has been described in connection with specific embodiments thereof, it will be understood that it is capable of further modifications and this application is intended to cover any variations, uses, or adaptations, including such departures from the present disclosure as come within known or customary practice within the art and as may be applied to the essential features hereinbefore set forth, and as follows in the scope of the appended claims.

What is claimed is:

1. A surface functionalized with cross linking groups, the surface adapted to receive antibodies and/or fragments thereof that bind a specific subtype of cells capturing the cells onto the functionalized surface and/or that bind cellular products, the surface comprising:
an antibody binding biomolecule comprising a linker region which is covalently crosslinked to functional groups on the surface and an antibody binding region that binds to any non-variable region of the antibodies or the fragments thereof and controls the orientation of the antibodies and/or the fragments thereof such that the variable regions are away from the surface, and the antibody binding biomolecule having a molecular weight of less than 10,000 g/mol; and
a cell interacting biomolecule comprising a linker region which is covalently crosslinked to functional groups of the surface and a cell interacting region, the cell interacting biomolecule has a three dimensional structure such that the cell interacting region is oriented away from the surface when the linker region has crosslinked with the surface, and the cell interacting biomolecule has a molecular weight of less than 10,000 g/mol;
wherein a concentration of the antibody binding biomolecule and a concentration of the cell interacting biomolecule on the surface are each independently controlled within a predetermined range that allows both the antibodies and/or the fragments thereof and the cell binding biomolecule to be functional.

2. The surface of claim 1, wherein the molecular weight of the antibody binding biomolecule is between about 200 g/mol to about 2000 g/mol and the molecular weight of the cell interacting biomolecule is between about 200 g/mol to about 2000 g/mol.

3. (canceled)

4. The surface of claim 1, wherein the antibody binding biomolecule and/or the cell interacting biomolecule further comprises one or more spacing regions comprising polyethylene glycol (PEG) and/or glycine.

5. (canceled)

6. The surface of claim 1, wherein the cell interacting region of the cell interacting biomolecule comprises a peptide derived from extracellular matrix proteins.

7. The surface of claim 1, wherein the antibody binding region of the antibody binding biomolecule comprises a RRGW peptide.

8. The surface of claim 1, further comprising a cross-linker binds the antibody binding biomolecule and/or the linker region of the cell interacting biomolecule to the surface, preferably comprising sulfosuccinimidyl 4-(n-maleimidophenyl)butyrate (Sulfo-SMPB), sulfosuccinimidyl 4-(-N-maleimidomethyl)cyclohexane-1-carboxylate (Sulfo-SMCC) or other amine-to-sulfhydryl bi-functional linkers.

9. (canceled)

10. The surface of claim 1, wherein the cell interacting region of the cell interacting biomolecule comprises a RGD peptide.

11. The surface of claim 1, wherein the surface is one of a flat surface, an interior cylindrical surface, an helix/screw-shaped material or the surface of a microbead.

12. The surface of claim 1, wherein the surface is a slide, a multiwell plate, a flask or microcarrier, or a bioreactor.

13. The surface of claim 1, wherein the surface is of glass, polystyrene, poly propylene, polytetrafluoroethylene, fluorinated ethylene propylene a metal or metal alloy such as cobalt-chrome (CoCr).

14. The surface of claim 1, wherein a ratio of the molecular weight of the antibody binding biomolecule to the molecular weight of the cell interacting biomolecule is between about 1:10 to about 10:1.

15. The surface of claim 1, wherein the cells are cancer cells, primary cells or in vivo cells.

16. The surface of claim 1, wherein the cells are endothelial progenitor cells.

17. The surface of claim 16, wherein the endothelial progenitor cells are endothelial colony-forming cells.

18. (canceled)

19. (canceled)

20. A method of producing a surface as defined in claim 1 having a dual function of capturing cells and/or cell products and promoting proliferation, adhesion, and/or spreading of the cells, comprising:
a) providing an activated surface having a crosslinking chemical group bound to one or more linker molecules;
b) crosslinking an antibody binding biomolecule to the one or more linking arms at the linker region of the antibody binding biomolecule by adding a predetermined concentration of the antibody binding biomolecule onto the surface, the antibody binding biomolecule having an antibody binding region that binds to the Fc region of antibodies and/or the fragments thereof and controls the orientation of the antibodies and/or the fragments thereof such that the variable regions are away from the surface, and the antibody binding biomolecule having a molecular weight of less than 10,000 g/mol, and
crosslinking a cell interacting biomolecule to the one or more linkers at the linker region of the cell interacting biomolecule by adding a predetermined concentration of the cell interacting biomolecule onto the surface, the cell interacting biomolecule having a cell interacting region that interacts with cellular receptors of the cells to promote the adhesion, spreading and/or proliferation of the cells on the surface, a three dimensional structure such that the cell interacting region is oriented away from the surface when the linker region has crosslinked with the surface, and a molecular weight of less than 10,000 g/mol,
thereby obtaining a conjugated surface; and
c) incubating the conjugated surface with the antibodies and/or the fragments thereof and allowing the antibodies and/or the fragments thereof to bind the antibody binding biomolecule.

21. The method of claim 20, further comprising prior to step a) an optimization step to determine the predetermine concentration of the antibody binding biomolecule and the predetermined concentration of the cell interacting biomolecule such that the surface has an optimized dual function of both capturing the cells and promoting the proliferation, adhesion, and/or spreading of the cells.

22. The method of claim 20, further comprising a first washing step between step b) and step c) and a second washing step after step c).

23. The method of claim 20, further comprising prior to step a), activating an inert surface to obtain the activated surface.

24. The method of claim 20, wherein the molecular weight of the antibody binding biomolecule and/or the molecular weight of the cell interacting biomolecule is between about 200 g/mol to about 2000 g/mol.

25. (canceled)

26. The method of claim 20, wherein the antibody binding biomolecule and/or the cell interacting biomolecule further comprises one or more spacing regions comprising polyethylene glycol (PEG) and/or glycine.

27-39. (canceled)
